(19) United States
(12) Patent Application Publication
(10) Pub. No.: US 2016/0279399 A1
(43) Pub. Date: Sep. 29, 2016

(54) OSMOTIC DRUG DELIVERY DEVICES, KITS, AND METHODS

(71) Applicant: TARIS BIOMEDICAL LLC, Lexington, MA (US)

(72) Inventors: Heejin LEE, Bedford, MA (US); Karen DANIEL, Newton, MA (US)

(21) Appl. No.: 15/033,692

(22) PCT Filed: Nov. 5, 2014
(86) PCT No.: PCT/US2014/064063

§ 371 Date: May 2, 2016

Related U.S. Application Data
(60) Provisional application No. 61/899,982, filed on Nov. 5, 2013.

(51) Int. Cl. A61M 31/00; A61L 31/16; A61K 31/7068; A61L 31/10; A61K 9/20; A61K 9/00; A61L 31/06

(52) U.S. Cl. CPC A61M 31/002; A61K 9/0004; A61L 31/16; A61L 31/06; A61L 31/10; A61K 9/20; A61K 9/2013; A61K 31/7068; A61K 9/2018; A61K 9/2009; A61M 2205/04; A61M 2209/045; A61J 1/2096

(57) ABSTRACT

Medical devices, kits, and methods are provided for delivering a fluid containing a drug to a patient. Devices (102, 402, 702) include a housing (104, 404, 704) defining a lumen and an osmotically-driven piston (420) moveable within the lumen. The housing may be elastically deformable between a first shape suitable for insertion through a patient's urethra and a second shape suitable for retention of the device in the patient's bladder.

### CROSS-REFERENCE TO RELATED APPLICATIONS

This application claims priority to U.S. Provisional Application No. 61/899,982, filed on Nov. 5, 2013, which is incorporated by reference herein in its entirety.

### BACKGROUND

This disclosure is generally in the field of drug delivery devices, and more particularly is in the field of drug delivery devices, kits, and methods that utilize an osmotic pressure to control release of drug to a patient.

Known methods and devices to osmotically deliver liquid drug formulations include syringe-type devices that utilize a plunger with an elastomeric piston positioned within a straight, rigid barrel. For example, the DUROS® drug-dispensing system has a piston made of elastomeric materials and rigid titanium housing. These devices experience a significant friction force that must be overcome to move the solid piston within in the syringe barrel even when the barrel is lubricated with a silicone or polydimethylsiloxane (PDMS) fluid. The rigidity of the device body also limits the sites in the patient in which such devices can be deployed, especially over an extended period without patient pain or discomfort.

U.S. Pat. No. 8,182,464 to Lee et al. and U.S. Pat. No. 8,343,516 to Daniel et al. describe drug delivery devices and methods for local administration of drug to the bladder. U.S. Application Publication No. 2011/0060309 and U.S. Pat. No. 8,679,094 by TARTS Biomedical also describe various drug delivery devices that provide controlled release of drug from a flexible housing. These flexible devices advantageously may be freely and tolerably retained in a patient's bladder while releasing drug over an extended period. Embodiments of these devices that employ osmotic pressure to drive out the drug have no piston and rely, at least in part, on the formulation of the solubilized drug in the device to create the osmotic pressure driving force. In this way, any osmotic agent, which may be necessary for certain low solubility drugs, is released from the device with the drug.

It would be desirable, however, to provide new osmotically driven drug delivery systems wherein the formulation of the solubilized drug in the device can be primarily selected independently from design considerations for producing the osmotic pressure driving force. It would also be desirable to provide such drug delivery devices and methods that are suitable for use in the bladder.

### SUMMARY

In one aspect, a medical device is provided that includes a housing defining a lumen and an osmotically-driven piston moveable within the lumen. In certain embodiments, the housing is elastically deformable between a first shape suitable for insertion through a patient's urethra and a second shape suitable for retention of the device in a patient's bladder.

In another aspect, a kit is provided that includes a medical device as described herein, a container housing a fluid (or a precursor thereof) to be delivered to a patient, and a device for transferring the fluid (or precursor) from the container and into the medical device.

In yet another aspect, a method of drug delivery is provided, which includes deploying into a patient's bladder via the patient's urethra a drug delivery device having a housing defining a lumen and a fluid to be dispensed. In certain embodiments, the device is elastically deformable between a first shape suitable for insertion through the urethra and a second shape suitable for retention of the device in the bladder, and the device is operable to move an osmotically-driven piston within the lumen to displace the fluid from the device.

### BRIEF DESCRIPTION OF THE DRAWINGS

FIG. 1 illustrates a kit including a cross-sectional view of a medical device, in accordance with one embodiment described herein.

FIG. 2 is a cross-sectional view of a plug, which may be used with an embodiment of the medical devices described herein.

FIG. 3A illustrates a kit including a cross-sectional view of a medical device during filling, in accordance with one embodiment described herein.

FIG. 3B is a cross-sectional view of the medical device of FIG. 3A after filling.

FIG. 3C is a cross-sectional view of the medical device of FIG. 3A after plugging the air vent and release structure.

FIG. 4A is a cross-sectional view of a medical device after filling, in accordance with one embodiment described herein.

FIG. 4B is a cross-sectional view of the medical device of FIG. 4A during dispensing of the fluid from the device.

FIG. 5A is a cross-sectional view of a medical device containing a solid or semi-sold drug formulation prior to device filling, in accordance with one embodiment described herein.

FIG. 5B is a cross-sectional view of the medical device of FIG. 5A during filling.

FIG. 5C is a cross-sectional view of the medical device of FIG. 5A after reservoir filling and plugging of the air vent.

FIG. 6A is a cross-sectional view of a medical device prior to filling, in accordance with another embodiment described herein.

FIG. 6B is a cross-sectional view of the medical device of FIG. 6A during filling.

FIG. 6C is a cross-sectional view of the medical device of FIG. 6A after filling.

FIG. 7 is a perspective and partial cut-away view of one embodiment of a medical device, in accordance with an embodiment described herein in a coiled configuration for bladder retention.

FIG. 8 is a cross-sectional view of a device housing with a single lumen, in accordance with one embodiment described herein.

FIG. 9 is a cross-sectional view of a device housing with multiple lumens, in accordance with another embodiment described herein.

FIG. 10 is a cross-sectional view of a comparative medical device tested in the Examples.

FIG. 11 is a cross-sectional view of a medical device tested in the Examples.

FIG. 12 is a cross-sectional view of a medical device tested in the Examples.

FIG. 13 is a graph showing percent gemcitabine released over time for devices with and without an air bubble piston.

FIG. 14 is a graph showing the gemcitabine release rate over time for devices with and without an air bubble piston.

FIG. 15 is a graph showing the percent citrate released over time for devices with and without an air bubble piston.

FIG. 16 is a graph showing the citrate release rate over time for devices with and without an air bubble piston.

FIG. 17 is a graph showing the percent gemcitabine released over time for devices with various osmotic agent formulations.

FIG. 18 is a graph showing the gemcitabine release rate over time for devices with various osmotic agent formulations.

FIG. 19 is a graph showing the percent urea released over time for devices with various osmotic agent formulations.

FIG. 20 is a graph showing the urea release rate over time for devices with various osmotic agent formulations.

### DETAILED DESCRIPTION

In one aspect, osmotically driven drug delivery devices, methods, and kits are provided herein. The devices may be configured to deliver liquid drug formulations via an osmotically driven, flexible fluid piston. The piston is a gas or liquid that is substantially immiscible in either the osmotic solution on the driving side of the piston and/or the liquid drug formulation on the dispensing side of the piston. In one embodiment, the piston is a bubble, or slug, of air or another gas. In the examples and figures, the fluid piston consists of air and is sometimes referred to as an “air gap” or “air bubble.” In one embodiment, the fluid piston comprises a gel or suspension. The piston preferably is substantially non-reactive with the liquid drug formulation and/or with the osmotic solution.

Advantageously, because the piston is a fluid, it can conform to the shape of the flexible drug reservoir, which is the elongated channel, compartment, or housing in which the drug formulation is stored until displaced by the piston. In this way, the flexible fluid piston advantageously enables the system to be bent, kinked, or distorted without failure in drug delivery or leakage at the piston. In addition, because the flexible fluid piston is near-frictionless, advancement of the piston is beneficially more responsive. For instance, the piston advancement may be significantly faster than that of a conventional syringe system with a solid, elastomeric piston under the same osmotic pressure. Furthermore, it may also be advantageous that the osmotic agent is not released into the patient with the drug.

In another aspect, a medical device is provided that includes: (i) a housing defining a lumen; and (ii) an osmotically-driven piston moveable within the lumen, wherein the housing is elastically deformable between a first shape suitable for insertion through the patient's urethra and a second shape suitable for retention of the device in the patient's bladder. In an embodiment, the medical device further includes (iii) a substance to be dispensed to a patient, wherein the device is operable to move the piston within the lumen to displace the substance from the device.

In a particular embodiment of the medical device, the housing comprises an elongated tube, the piston comprises a gas, and the substance comprises a drug. While certain embodiments are described with reference to the drug containing portion of the housing being an elongated tube, it should be understood that other suitable housing designs may also be used.

As used herein, the term “substance” may refer to a fluid drug formulation to be delivered to a patient or a precursor of the fluid drug formulation to be delivered to a patient (e.g., a solid or semi-solid drug formulation, a solvent for a solid or semi-solid drug formulation). For example, the drug formulation may be provided in a dry solid form for stable storage of the active pharmaceutical ingredient prior to use, and then immediately before use, the drug formulation is reconstituted, i.e., solubilized, by injection of a pharmaceutically acceptable vehicle, e.g., saline or another biocompatible liquid optionally comprising one or more pharmaceutically acceptable excipients.

The devices and methods disclosed herein may be adapted for use in humans, whether male or female, adult or child, or for use in animals, such as for veterinary or livestock applications. Accordingly, the term “patient” may refer to a human or other mammalian subject.

The devices, kits, and methods disclosed herein may build upon various features of the drug delivery devices and methods described in U.S. Pat. No. 8,182,464 (MIT 11824 DIV), U.S. Pat. No. 8,343,516 (TB 102), U.S. Pat. No. 8,679,094 (TB 112), U.S. Pat. No. 8,690,840 (TB 117), U.S. Pat. No. 8,721,621 (TB 107), as well as in U.S. Patent Application Publications No. 2009/0149833 (MIT 12988), No. 2010/0331770 (TB 101), No. 2011/0060309 (TB 108), No. 2012/0089121 (TB 116), No. 2012/0191068 (TB 120), No. 2013/0158675 (TB 113), and No. 2014/0276636 (TB 134), each of which is incorporated by reference herein in pertinent part.

Various non-limiting embodiments and features of the medical devices, methods, and kits are described in detail below.

Drug Delivery Devices

The device may be provided with the drug formulation stored on-board from the point of manufacture, or a fluid drug formulation or a precursor thereof can be loaded into the device before insertion into a patient.

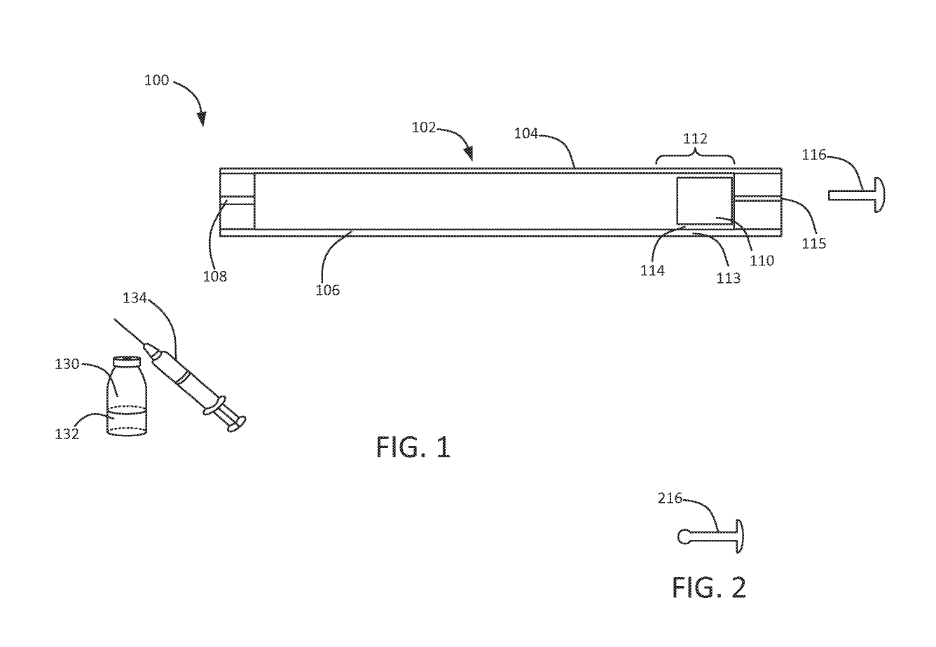

Therefore, in an embodiment ready for loading with drug, as shown in FIG. 1, the device 102 includes a housing 104 comprising an elongated tube 106 with a first end having a release structure 108 for releasing the fluid and an opposed second end. The elongated tube 106 is configured to receive a fluid drug or a precursor thereof. The housing 104 also defines a reservoir 114 that is connected to the second end of the elongated tube 106 and in which an osmotic agent 110 is disposed. The housing 104 includes a water permeable wall 112 for permitting water to enter the reservoir 114 and contact the osmotic agent 110. The device 102 is configured such that upon receipt of the fluid or the precursor thereof, the piston comprises a gas formed between the fluid and the osmotic agent 110. The device is configured to imbibe water into the reservoir 114 via the water permeable wall 112 to advance the gas piston through the elongated tube 106 via osmotic pressure generated by the osmotic agent to drive the fluid from the device via the release structure 108.

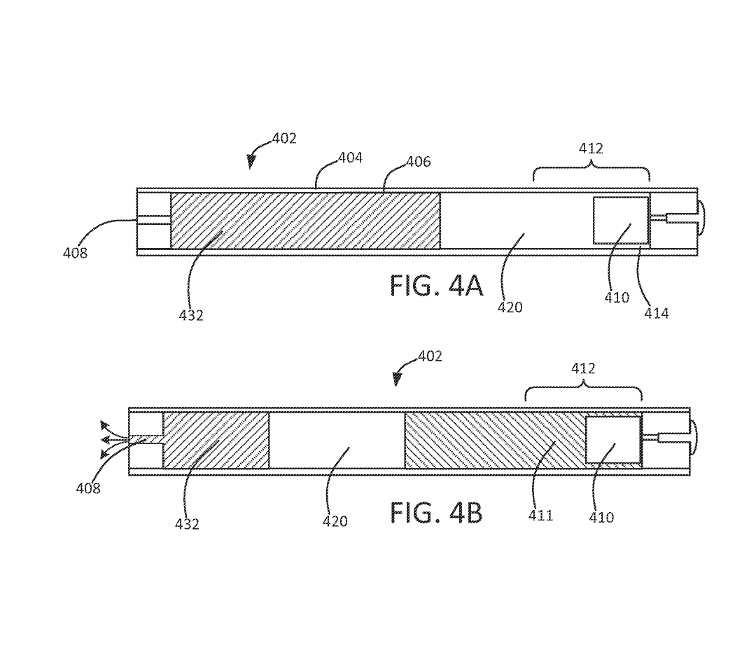

In an embodiment pre-loaded with the fluid, as shown in FIGS. 4A-4B, the device 402 includes a housing 404 comprising an elongated tube 406 with a first end having a release structure 408 for releasing the fluid 432 and an opposed second end. The housing 404 further defines a reservoir 414 that is connected to the second end of the elongated tube 406 and in which an osmotic agent 410 is disposed. The housing includes a water permeable wall 412 for permitted water to enter the reservoir and contact the osmotic agent. As shown in FIG. 4B, gas piston 420 is operable to be advanced in the lumen of the elongated tube 406 toward the first end of the elongated tube 406 under osmotic pressure generated by the osmotic agent 410 to cause the fluid 432 to be displaced out of the lumen via the release structure 408.

In these embodiments, as shown in FIGS. 4A-4B, the device is configured to imbibe water 411 via the water permeable wall 412, such that an osmotic pressure is developed within the device which causes the piston 420 to be advanced to drive the drug-containing fluid 432 from the device 402. For example, the device may be configured for insertion or implantation in a patient at a site, such as a body lumen, in which an aqueous bodily fluid is present. For example, the device may be configured for insertion into the bladder, where urine may be imbibed into the device to effectuate release of the fluid drug formulation.

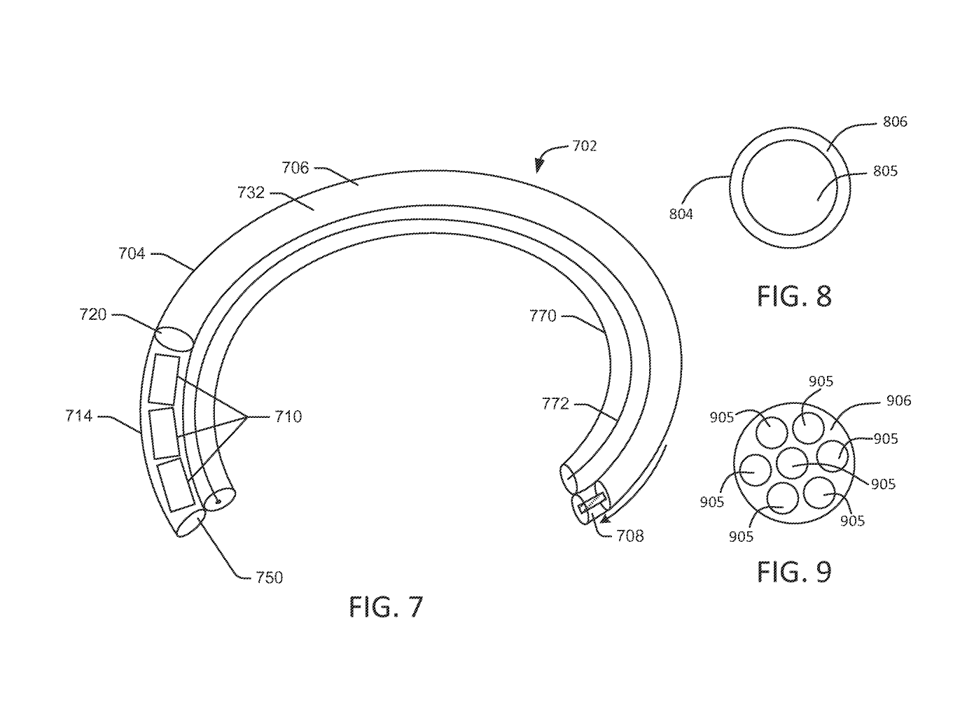

As shown in FIG. 8, in certain embodiments, the housing 804 comprises an annular tube 806 having a single, central lumen 805. In another embodiment, the elongated tube 906 includes a multiple lumens 905, as shown in FIG. 9. Each lumen may be configured to receive, or may be loaded with, a fluid drug formulation or a precursor thereof (e.g., a solvent for the drug).

As shown in FIG. 1, in certain embodiments, the reservoir 114 is formed, or defined, by an annular tube 113 integrally formed with the elongated tube 106 which contains or is configured to receive the fluid. In one embodiment, the housing has a single tube defining a first compartment (e.g., a drug fluid containing compartment) and a second compartment (e.g., osmotic agent containing compartment).

In other embodiments, the reservoir is formed by an annular tube that is connected to the elongated tube which contains or is configured to receive the fluid. In one embodiment, the device includes a connector connecting the elongated tube and the reservoir. For example, the connector may be a spacer orifice, valve, or other suitable connection mechanism. For example, the connector may be a barbed polypropylene fitting.

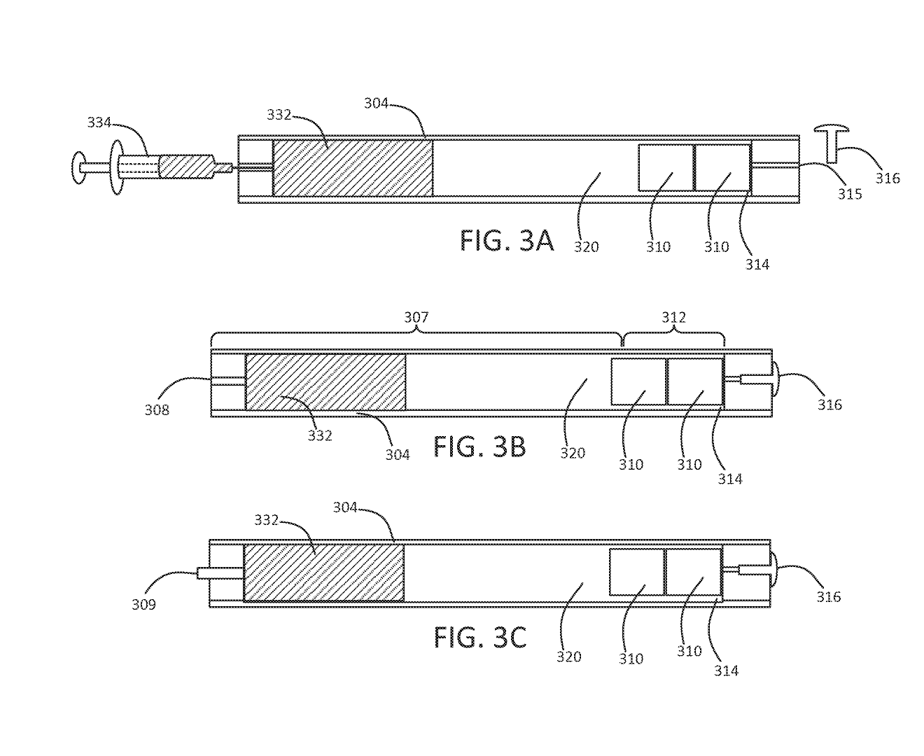

As shown in FIGS. 3A-3C, upon receipt of the fluid or precursor 332 in the elongated tube 304, a gas piston 320 (a slug or bubble of air) is formed between the fluid 332 and the osmotic agent 310. The gas piston 320 is interposed between the osmotic agent 310 and the fluid drug formulation 332 and is operable to be advanced toward the release structure 308 (i.e., the first end of the elongated tube) under osmotic pressure generated by the osmotic agent 310 to cause the fluid drug formulation 332 to be displaced out of the device via the release structure.

In one embodiment, the wall of the elongated tube and/or the wall of the reservoir are formed of a polymer, such as an elastomeric polymer having a hardness ranging from 50 Shore A to 90 Shore A. For example, the polymer may be silicone or polyurethane. In one embodiment, as shown in FIGS. 3A-3C, the wall 307 of the elongated tube 304 is water impermeable. In certain embodiments, a portion 307 of the wall of the reservoir 314, other than the water permeable portion 312, is also water impermeable. In one embodiment, the wall of the elongated tube and/or the wall of the reservoir are also air impermeable. For example, the elongated tube and/or reservoir may be at least partially formed of an elastomeric polymer that is substantially water and gas impermeable or has a coating that is substantially water and gas impermeable. For example, the wall of the elongated tube and/or the wall of the reservoir may be formed of a parylene coated silicone. In one embodiment, the parylene is parylene C.

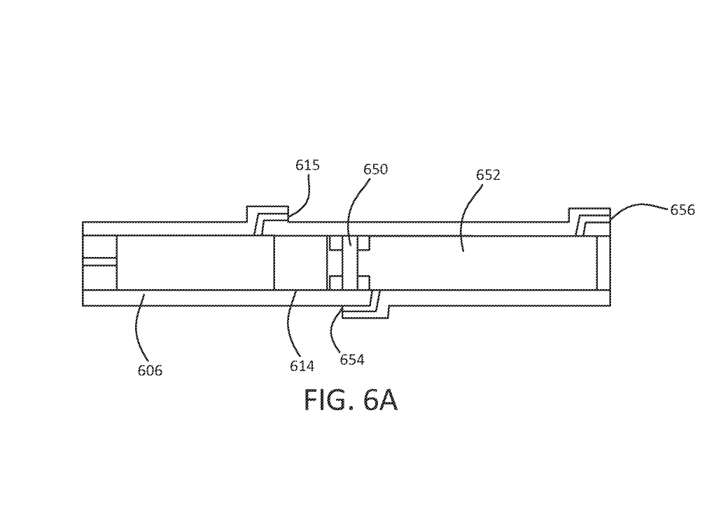

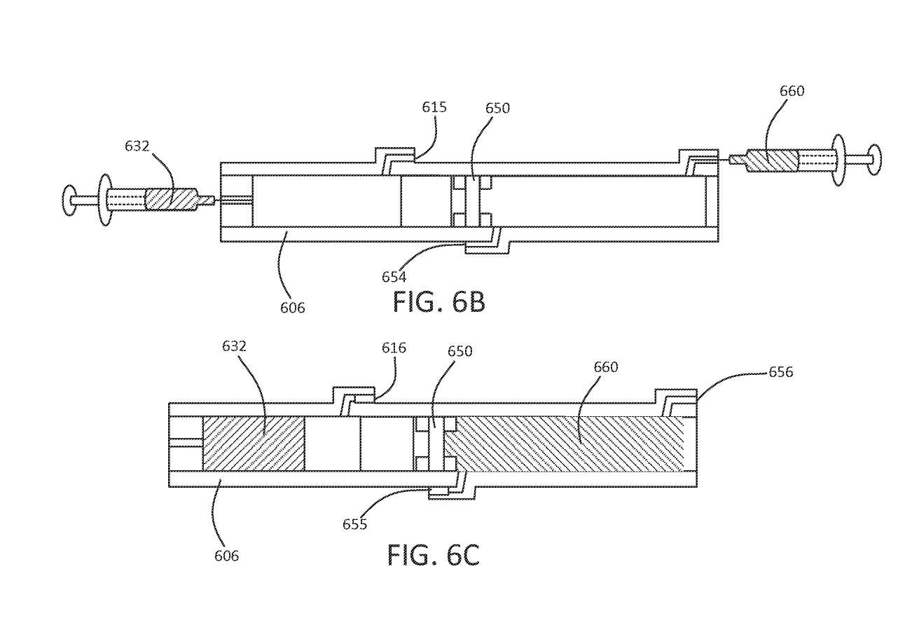

In one embodiment, the reservoir, or housing, which contains the osmotic agent, is a water permeable tube. For example, as shown in FIG. 1, the reservoir 114 may be a tube having a water permeable wall region 112. In another embodiment, as shown in FIGS. 6A-6C, the water permeable portion of the wall of the reservoir 614 includes a water permeable membrane 650 at one end of the reservoir 614. For example, as shown in FIG. 7, the reservoir 714 may be tubular and include a water permeable disc 750 at an end of the tube. For example, the water permeable portion of the wall of the reservoir may include hydrophilic polymers, thermoplastic polyurethane, such as Tecophilic® (Lubrizol Advanced Materials, Inc.), HydroThane™ (AdvanSource Biomaterials), Quadraphilic™ (Biomerics), or hydrophilic polyether block amide copolymers, such as hydrophilic Pebax® MV 1074 SA 01 MED (Arkema).

In one embodiment, the elongated tube that contains or receives the fluid has an inner diameter sized such that capillary force is dominant over gravitational force within the tube. That is, the tube may be sized and shaped such that the fluid drug formulation is able to flow through the tube toward the dispensing end substantially without the assistance of gravity. Cross-sectional views of a single lumen tube and a multi-lumen tube are shown in FIGS. 8 and 9, respectively. If the total opening area of the multi-lumen tube is the same as that of a single lumen tube, the multi-lumen tube may be preferable to provide reliable separation of the fluid drug formulation, fluid piston, and osmotic solution. The inner diameter of each individual lumen should be small enough so that capillary force can dominate over buoyant or gravitational force. Then, a compressed air slug will remain separated from the fluid drug formulation and can act as a piston or plunger supported by the osmotic influx. As compared with FIG. 8, the tube of FIG. 9 has multiple small capillary channels (i.e., lumens) 905 that can serve as a pathway for the fluid formulation.

For capillary force to be dominant over buoyancy/gravity, a dimensional analysis can be performed based on the Bond number, which is represented by:

$$Bo = \frac{ρ {aL}^{2}}{γ} .$$

Generally, the Bond number measures the effect of surface tension forces compared to body (gravitational) forces. A high Bond number indicates that the system is relatively unaffected by surface tension effects while a low number (typically less than one) indicates that surface tension dominates. Analyzing a device having a Bond number significantly less than 1 (Bo<<1) gives L<<\sqrt{γ/(ρ/g)}=2.67 mm, where γ=0.07 N/m (surface tension of the interface of water in contact with air), ρ=1 g/cc g=9.8 m/s², and L is a characteristic length scale, i.e., a tube inner diameter where a tubular housing is used. Thus, in certain embodiments, the tube has an inner diameter of less than 2.67 mm, for example from 1.52 mm to 2.64 mm. Also, if water is mixed with other molecules, such as NaCl or sucrose (both can be used as osmotic agents), the surface tension will be 0.083 N/m for NaCl 6.0M aqueous solution at 20° C. and 0.076 N/m for sucrose 55% w/w aqueous solution at 20° C. The higher surface tensions will help the serial distribution of the compressed air slug, and two fluid regions in the tube.

In one embodiment, the elongated tube and the reservoir are formed of a silicone tube having an inner diameter of about 1 mm to about 3 mm. For example, the housing may have a central lumen with a diameter between 1 mm and 3 mm.

In one embodiment, as shown in FIGS. 3A-3C, the device also includes an air vent 315 in fluid communication with the elongated tube or the reservoir 314 (illustrated in communication with reservoir 314). The air vent 315 is configured to be plugged, such as by plug 316, once the elongated tube receives the fluid or precursor 332. During fluid filling, as in FIG. 3A, the air vent 315 may remain open so that the fluid cannot be expelled (by the gas piston) after filling. Since the air vent 315 may be positioned, in this embodiment, behind one or more osmotic tablets 310, the tablet(s) 314 should be dimensioned and shaped to avoid creating a seal in the reservoir 314 and thereby to permit air to flow around the tablet(s) 310 toward the air vent 315 during filling.

In an alternative embodiment, the air vent is temporarily defined and the plug omitted. That is, the end plug may be formed of an elastic material through which a hollow needle can be inserted to provide a passage through which air can be vented during the filling process, and after filling, the hollow needle can be withdrawn to permit the elastic material to self-seal the hole made by the hollow needle. In this way, no plug is needed.

In one embodiment, the fluid release structure includes an orifice and/or a check valve. For example, a check valve may prevent capillary or unnecessary back diffusion from outside to inside of the device. For example, as shown in FIG. 3A, the device may be configured to receive the fluid or precursor 332 via the release structure 308, such as via a syringe 334.

As shown in FIG. 12, in one embodiment, the device includes two compartments 1206 loaded with the fluid drug formulation 1232 and connected by a spacer orifice connector 1282.

In one embodiment, as shown in FIGS. 6A-6C, the device also includes a compartment 652 adjacent to the reservoir 614 and configured to house water 660 to be imbibed into the reservoir via the water permeable portion 650 of the wall of the reservoir. For example, devices having an on-board water compartment may be suitable for use at water-scarce tissue sites for drug delivery, such as in the uterus, in a patient.

In certain embodiments, as shown in FIGS. 6A-6C, the water permeable wall 650 of the reservoir 614 includes a hydrophilic membrane positioned between the reservoir 614 and the compartment 652. In one embodiment, the device further includes an air vent 654 in fluid communication with the compartment 652. The air vent, in this embodiment, is configured to be plugged, such as with plug 655, once the compartment receives the water 660. The compartment may also include a port 656 through which water 660 may be introduced into the compartment. The port 656 may be left open so that the compartment 652 does not collapse while water is drawn into the osmotic reservoir 614 from the compartment 652. In another embodiment, the wall of the compartment can be made of collapsible material, such as thin plastic film, so the wall can be readily collapsed while water is drawn into the osmotic reservoir from the compartment. In this case, any port in the compartment is not left open upon receipt of the water.

As shown in FIGS. 6A-6C, in certain embodiments, the device also includes an air vent 615 in fluid communication with the elongated tube 606 or the reservoir 614. The air vent 615 is configured to be plugged, such as by plug 616, once the elongated tube 606 receives the fluid or precursor 632. During fluid filling, as in FIG. 6B, the air vent 615 may remain open so that the fluid cannot be expelled (by the gas piston) after filling.

In one embodiment, the device includes a first compartment for housing the drug solution, a second compartment housing the osmotic agent, and a third compartment also for receiving and releasing a fluid. For example, a device may have a dual release design with an osmotic region in the center of the device and multiple air slug/drug compartments adjacent thereto.

In one embodiment, as shown in FIG. 7, a drug delivery device 702 includes: (i) an elongated flexible tube 706 having a lumen therein loaded with a liquid drug formulation 732, the tube having (a) a first end having a dispensing aperture 708 for releasing the liquid drug formulation 732 and (b) an opposed second end; (ii) a housing portion 714 connected to the second end of the elongated tube 706 and defining a reservoir in which an osmotic agent 710 is disposed, the housing portion having a water permeable wall 750 for permitting water to enter the reservoir and contact the osmotic agent 710; and (iii) a fluid piston 720 in the lumen interposed between the osmotic agent 710 and the liquid drug formulation 732, wherein the fluid piston 720 is operable to advance in the lumen toward the first end under osmotic pressure generated by the osmotic agent 710 to cause the liquid drug formulation 732 to be displaced (in the direction of the arrow) out of the lumen via the dispensing aperture 708. In use, water is imbibed through wall 750, enters the lumen and solubilizes the osmotic agent 710 to form an osmotic solution. Water continues to be imbibed, creating an osmotic pressure, which is relieved by displacement of the fluid piston 720.

Device 702 further includes a retention frame lumen 770 in which a retention frame 772 is secured. As shown in FIG. 7, the retention frame, which may comprise an elastic wire (e.g., a superelastic alloy such as nitinol), imparts a coiled shape to the device. In the illustrated embodiment, the retention frame urges the medical device into a shape which comprises a coil in the absence of a compressive load. For example, this shape would be suitable for retention of the device in the patient's bladder, in contrast to the device embodiment shown in FIG. 11, wherein a compressive load holds the medical device in the straightened shape shown, which would be suitable for insertion of the device through a lumen in the patient's urethra.

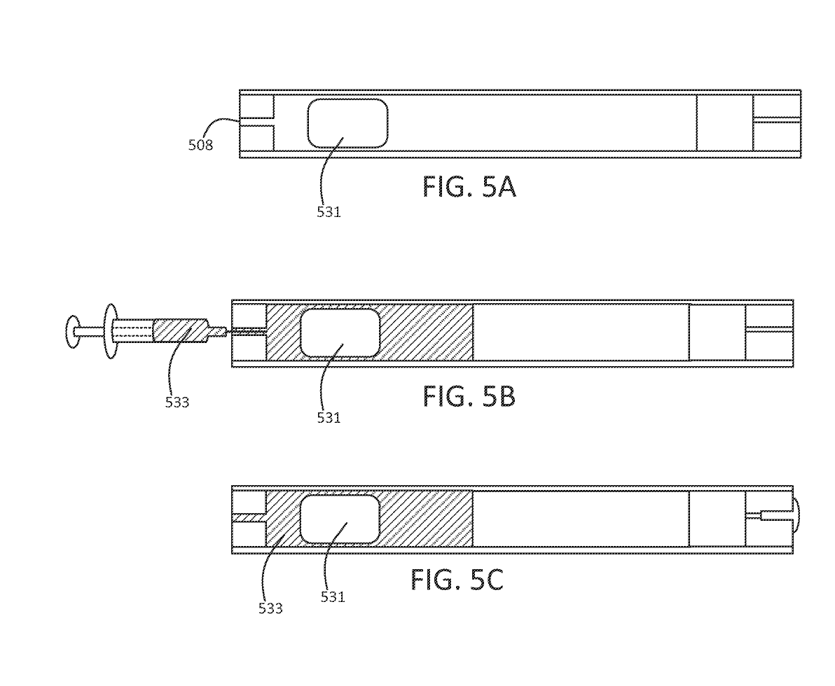

In one embodiment, as shown in FIG. 1, the fluid that is loaded into the elongated tube is a solution of the drug, i.e., it is the fluid drug formulation to be released. In another embodiment, as shown in FIGS. 5A-5C, a solid or semi-solid formulation of the drug 531 is housed within the elongated tube, and the fluid 533 that is loaded into the elongated is a precursor for the fluid drug formulation (e.g., a solvent for the drug formulation), such that upon receipt of the fluid precursor in the elongated tube, the solvent dissolves the drug to form the fluid drug formulation to be released from the device. For example, the drug may be in the form of a powder or one or more tablets, capsules, or pellets. The solvent may be, for example, water, dimethyl sulfoxide (DMSO) and/or dimethyl formamide (DMF). In a particular embodiment, DMSO may be the preferred solvent, because it is already known for use as an intravesical agent to relieve the symptoms of the bladder condition called interstitial cystitis.

The term “drug” as used herein encompasses any suitable pharmaceutically active ingredient. The drug may be small molecule, macromolecule, biologic, or metabolite, among other forms/types of active ingredients. The drug described herein includes its alternative forms, such as salt forms, free acid forms, free base forms, and hydrates. The drug may be formulated with one or more pharmaceutically acceptable excipients known in the art. Non-limiting examples of the drug include gemcitabine, oxaliplatin, and/or another chemotherapeutic agent; oxybutynin, trospium, and/or another antimuscarinic agent; and/or lidocaine and/or another anesthetic agent. In one embodiment, the first compartment (e.g., the elongated tube) may be loaded with two or more types of drug tablets (e.g., different drugs), so that a combination of drugs may be delivered.

In some embodiments, the drug is one used to treat pain. A variety of anesthetic agents, analgesic agents, and combinations thereof may be used. In one embodiment, the drug is an anesthetic agent. The anesthetic agent may be a cocaine analogue. The anesthetic agent may be an aminoamide, an aminoester, or combinations thereof. Representative examples of aminoamides or amide-class anesthetics include articaine, bupivacaine, carticaine, cinchocaine, etidocaine, levobupivacaine, lidocaine, mepivacaine, prilocalne, ropivacaine, and trimecaine. Representative examples of aminoesters or ester-class anesthetics include amylocalne, benzocaine, butacaine, chloroprocaine, cocaine, cyclomethycaine, dimethocaine, hexylcaine, larocaine, meprylcaine, metabutoxycaine, orthocaine, piperocaine, procaine, proparacaine, propoxycaine, proxymetacaine, risocaine, and tetracaine. The drug also can be an antimuscarinic compound that exhibits an anesthetic effect, such as oxybutynin or propiverine. In embodiments, the analgesic agent includes an opioid. Representative examples of opioid agonists include alfentanil, allylprodine, alphaprodine, anileridine, benzyl morphine, bezitramide, buprenorphine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene fentanyl, heroin, hydrocodone, hydromorphone, hydroxypethidine, isomethadone, ketobemidone, levorphanol, levophenacylmorphan, lofentanil, meperidine, meptazinol, metazocine, methadone, metopon, morphine, myrophine, nalbuphine, narceine, nicomorphine, norlevorphanol, normethadone, nalorphine, normorphine, norpipanone, opium, oxycodone, oxymorphone, papavereturn, pentazocine, phenadoxone, phenomorphan, phenazocine, phenoperidine, piminodine, piritramide, proheptazine, promedol, properidine, propiram, propoxyphene, sufentanil, tilidine, tramadol, pharmaceutically acceptable salts thereof, and mixtures thereof. Other opioid drugs, such as mu, kappa, delta, and nociception opioid receptor agonists, are contemplated. Representative examples of other suitable pain relieving agents include such agents as salicyl alcohol, phenazopyridine hydrochloride, acetaminophen, acetylsalicylic acid, flufenisal, ibuprofen, indoprofen; indomethacin, naproxen.

In some embodiments, the drug is one used to treat inflammatory conditions such as interstitial cystitis, radiation cystitis, painful bladder syndrome, prostatitis, urethritis, post-surgical pain, and kidney stones. Non-limiting examples of drugs for these conditions include lidocaine, glycosaminoglycans (e.g., chondroitin sulfate, sulodexide), pentosan polysulfate sodium (PPS), dimethyl sulfoxide (DMSO), oxybutynin, mitomycin C, heparin, flavoxate, ketorolac, or a combination thereof. Other non-limiting examples of drugs that may be used in the treatment of IC include nerve growth factor monoclonal antibody (MAB) antagonists, such as Tanezumab, and calcium channel alpha-2-delta modulators, such as PD-299685 or gabepentin.

In some embodiments, the drug is one used to treat urinary incontinence, frequency, or urgency, including urge incontinence and neurogenic incontinence, as well as trigonitis. Drugs that may be used include anticholinergic agents, antispasmodic agents, anti-muscarinic agents, β-2 agonists, alpha adrenergics, anticonvulsants, norepinephrine uptake inhibitors, serotonin uptake inhibitors, calcium channel blockers, potassium channel openers, and muscle relaxants. Representative examples of suitable drugs for the treatment of incontinence include oxybutynin, S-oxybutylin, emepronium, verapamil, imipramine, flavoxate, atropine, propantheline, tolterodine, rociverine, clenbuterol, darifenacin, terodiline, trospium, hyoscyamin, propiverine, desmopressin, vamicamide, clidinium bromide, dicyclomine HCl, glycopyrrolate aminoalcohol ester, ipratropium bromide, mepenzolate bromide, methscopolamine bromide, scopolamine hydrobromide, iotropium bromide, fesoterodine fumarate, YM-46303 (Yamanouchi Co., Japan), lanperisone (Nippon Kayaku Co., Japan), inaperisone, NS-21 (Nippon Shinyaku Orion, Formenti, Japan/Italy), NC-1800 (Nippon Chemiphar Co., Japan), Z D-6169 (Zeneca Co., United Kingdom), and stilonium iodide.

In some embodiments, the drug is one used to treat urinary tract cancer, such as bladder cancer and prostate cancer. Drugs that may be used include antiproliferative agents, cytotoxic agents, chemotherapeutic agents, or a combination thereof. Representative examples of drugs which may be suitable for the treatment of urinary tract cancer include Bacillus Calmette Guerin (BCG) vaccine, cisplatin, doxorubicin, valrubicin, gemcitabine, mycobacterial cell wall-DNA complex (MCC), methotrexate, vinblastine, thiotepa, mitomycin, fluorouracil, leuprolide, diethylstilbestrol, estramustine, megestrol acetate, cyproterone, flutamide, a selective estrogen receptor modulators (i.e. a SERM, such as tamoxifen), botulinum toxins, and cyclophosphamide. The drug may be a biologic, and it may comprise a monoclonal antibody, a TNF inhibitor, an anti-leukin, or the like. The drug also may be an immunomodulator, such as a TLR agonist, including imiquimod or another TLR7 agonist. The drug also may be a kinase inhibitor, such as a fibroblast growth factor receptor-3 (FGFR3)-selective tyrosine kinase inhibitor, a phosphatidylinositol 3 kinase (PI3K) inhibitor, or a mitogen-activated protein kinase (MAPK) inhibitor, among others or combinations thereof. Other examples include celecoxib, erolotinib, gefitinib, paclitaxel, polyphenon E, valrubicin, neocarzinostatin, apaziquone, Belinostat, Ingenol mebutate, Urocidin (MCC), Proxinium (VB 4845), BC 819 (BioCancell Therapeutics), Keyhole limpet haemocyanin, LOR 2040 (Lorus Therapeutics), urocanic acid, OGX 427 (OncoGenex), and SCH 721015 (Schering-Plough). Other intravesical cancer treatments include small molecules, such as Apaziquone, adriamycin, AD-32, doxorubicin, doxetaxel, epirubicin, gemcitabine, HTI-286 (hemiasterlin analogue), idarubicin, γ-linolenic acid, mitozantrone, meglumine, and thiotepa; large molecules, such as Activated macrophages, activated T cells, EGF-dextran, HPC-doxorubicin, IL-12, IFN-α2b, IFN-γ, α-lactalbumin, p53 adenovector, TNFα; combinations, such as Epirubicin+BCG, IFN+farmarubicin, Doxorubicin+5-FU (oral), BCG+IFN, and Pertussis toxin+cystectomy; activated cells, such as macrophages and T cells; intravesical infusions such as IL-2 and Doxorubicin; chemosensitizers, such as BCG+antifirinolytics (paramethylbenzoic acid or aminocaproic acid) and Doxorubicin+verapimil; diagnostic/imaging agents, such as Hexylaminolevulinate, 5-aminolevulinic acid, Iododexyuridine, HMFG1 Mab+Tc99m; and agents for the management of local toxicity, such as Formaline (hemorrhagic cystitis).

In some embodiments, the drug is one used to treat infections involving the bladder, the prostate, and the urethra. Antibiotics, antibacterial, antifungal, antiprotozoal, antiseptic, antiviral and other antiinfective agents can be administered for treatment of such infections. Representative examples of drugs for the treatment of infections include mitomycin, ciprofloxacin, norfloxacin, ofloxacin, methanamine, nitrofurantoin, ampicillin, amoxicillin, nafcillin, trimethoprim, sulfonamides trimethoprimsulfamethoxazole, erythromycin, doxycycline, metronidazole, tetracycline, kanamycin, penicillins, cephalosporins, and aminoglycosides.

In some embodiments, the drug is one used to treat fibrosis of a genitourinary site, such as the bladder or uterus. Representative examples of drugs for the treatment of fibroids include pentoxphylline (xanthine analogue), antiTNF, antiTGF agents, GnRH analogues, exogenous progestins, antiprogestins, selective estrogen receptor modulators, danazol and NSAIDs.

In some embodiments, the drug is one used to treat neurogenic bladder. Representative examples of such drugs include analgesics or anaesthetics, such as lidocaine, bupivacaine, mepivacaine, prilocalne, articaine, and ropivacaine; anticholinergics; antimuscarinics such as oxybutynin or propiverine; a vanilloid, such as capsaicin or resiniferatoxin; antimuscarinics such as ones that act on the M3 muscarinic acetylcholine receptor (mAChRs); antispasmodics including GABA_{B} agonists such as baclofen; botulinum toxins; capsaicins; α-adrenergic antagonists; anticonvulsants; serotonin reuptake inhibitors such as amitriptyline; and nerve growth factor antagonists. In various embodiments, the drug may be one that acts on bladder afferents or one that acts on the efferent cholinergic transmission, as described in Reitz et al., Spinal Cord 42:267-72 (2004).

In some embodiments, the drug is one used to treat incontinence due to neurologic detrusor overactivity and/or low compliant detrusor. Examples of these types of drugs include bladder relaxant drugs (e.g., oxybutynin (antimuscarinic agent with a pronounced muscle relaxant activity and local anesthetic activity), propiverine, impratroprium, tiotropium, trospium, terodiline, tolterodine, propantheline, oxyphencyclimine, flavoxate, and tricyclic antidepressants; drugs for blocking nerves innervating the bladder and urethra (e.g., vanilloids (capsaicin, resiniferatoxin), botulinum-A toxin); or drugs that modulate detrusor contraction strength, micturition reflex, detrusor sphincter dyssynergia (e.g., GABAb agonists (baclofen), benzodiazapines). In another embodiment, the drug is selected from those known for the treatment of incontinence due to neurologic sphincter deficiency. Examples of these drugs include α-adrenergic agonists, estrogens, β-adrenergic agonists, tricyclic antidepressants (imipramine, amitriptyline). In still another embodiment, the drug is selected from those known for facilitating bladder emptying (e.g., α-adrenergic antagonists (phentolamitie) or cholinergics). In yet another embodiment, the drug is selected from among anticholinergic drugs (e.g., dicyclomine), calcium channel blockers (e.g., verapamil) tropane alkaloids (e.g., atropine, scopolamine), nociceptin/orphanin FQ, and bethanechol (e.g., M3 muscarinic agonist, choline ester).

The osmotic agent may be in a solid, semi-solid, or solution form. In one embodiment, the osmotic agent is in the form of a powder or one or more tablets, capsules, or pellets. For example, a tubular reservoir may house one or more cylindrical osmotic agent tablets. The osmotic agent may be selected from the group consisting of: monosodium citrate, disodium citrate, trisodium citrate, lactose, sodium chloride, urea, sucrose, and combinations thereof. Other osmotic agents are also envisioned.

In a preferred embodiment, an example of which is shown in FIG. 7, the device 702 is elastically deformable. For example, the device may be elastically deformable between a first shape suitable for insertion through the patient's urethra and a second shape suitable for retention of the device in the patient's bladder. When in the retention shape after deployment in the bladder, for example, the device advantageously may resist excretion in response to the forces of urination or other forces. Since the devices are designed to be retained within a lumen or body cavity, they are capable of overcoming some of the deficiencies of conventional treatments, such as those related to the bladder.

The devices described herein can be inserted once and release drug over a desired period of time without surgery or frequent interventions. The devices, as a result, may reduce the opportunity for infection and side effects, may increase the amount of drug delivered locally or regionally to the bladder, and may improve the quality of life of the patient during the treatment process. After drug release is completed, the device is removed from the patient. Removal can be accomplished by a number of different methods, including retrieval by a physician, for example using a catheter or cystoscope, by a withdrawal using a retrieval string connected to the device which extends through the urethra, by having the device biodegrade or bioerode in the body, or by providing the device with a means to lose its retention shape so that the device (or parts thereof) can be excreted during urination. These means may include forming the device partially or entirely of bioerodible materials and/or by having the device lose buoyancy for example by permitting an entrapped gas to escape the device.

In one embodiment, the drug delivery device may naturally assume a retention shape and may be deformed, either manually or with the aid of an external apparatus, into a relatively straightened shape for insertion into the body. Once deployed the device may spontaneously or naturally return to the initial, retention shape for retention in the body. For the purposes of this disclosure, the term “retention shape” generally denotes any shape suited for retaining the device in the intended implantation location, including, but not limited to, a coiled or “pretzel” shape, which is suited for retaining the device in the bladder. Similarly, the term “relatively straightened shape” generally denotes any shape suited for deploying the drug delivery device into the body, including, but not limited to, a linear or elongated shape, which is suited for deploying the device through the working channel of catheter, cystoscope, or other deployment instrument positioned in a lumen of the body, such as the urethra.

In one embodiment, the drug delivery device does not need a retention frame to be elastically deformable between a relatively straightened shape and a retention shape. In these embodiments, the material from which the housing is formed makes the device capable of being elastically deformed between the two shapes. In another embodiment, the drug delivery device includes a retention frame 772 that is associated with the housing 704, for example in a separate lumen 770 housing the retention frame 772, such as shown in FIG. 7. The properties of the retention frame cause the device to function as a spring, deforming in response to a compressive load but spontaneously returning to its initial shape once the load is removed. In one embodiment, the retention frame 772 is located in a retention frame lumen 770 that is integrally formed or otherwise connected to the housing 704, as shown in FIG. 7. In another embodiment, the retention frame is affixed to the housing by suitable means, such as an adhesive.

In certain embodiments, the retention frame, like the devices themselves, may naturally assume the retention shape, may be deformed into the relatively straightened shape, and may spontaneously return to the retention shape upon insertion into the body. The retention frame in the retention shape may be shaped for retention in a body cavity, and the retention frame in the relatively straightened shape may be shaped for insertion into the body through the working channel of a deployment instrument such as a catheter or cystoscope. To achieve such a result, the retention frame may have an elastic limit, modulus, and/or spring constant selected to impede the device from assuming the relatively lower-profile shape once implanted. Such a configuration may limit or prevent accidental expulsion of the device from the body under expected forces. For example, the device may be retained in the bladder during urination or contraction of the detrusor muscle.

In one embodiment, the retention frame includes or consists of an elastic wire or an elastic strip. In one embodiment, the elastic wire may comprise a biocompatible shape-memory material or a biodegradable shape memory polymer as known in the art. For example, the retention frame may include a nitinol alloy wire. The elastic wire also may include a relatively low modulus elastomer, which may be relatively less likely to irritate or cause ulcer within the bladder or other implantation site and may be biodegradable so that the device need not be removed. Examples of low modulus elastomers include polyurethane, silicone, styrenic thermoplastic elastomer, and poly(glycerol-sebacate) (PGS). The elastic wire may be coated with a biocompatible polymer, such as a coating formed from one or more of silicone, polyurethane, styrenic thermoplastic elastomer, Silitek, Tecoflex, C-flex, and Percuflex.

The retention frame may have a two-dimensional structure that is confined to a plane, a three-dimensional structure, such as a structure that occupies the interior of a spheroid, or some combination thereof. The frames may comprise one or more loops, curls, or sub-circles, connected either linearly or radially, turning in the same or in alternating directions, and overlapping or not overlapping. The frames may include one or more circles or ovals arranged in a two-dimensional or a three-dimensional configuration, the circles or ovals, either closed or opened, having the same or different sizes, overlapping or not overlapping, and joined together at one or more connecting points. The retention frame portion also may be a three-dimensional structure that is shaped to occupy or wind about a spheroid-shaped space, such as a spherical space, a space having a prorate spheroid shape, or a space having an oblate spheroid shape. Retention frame portions may be shaped to occupy or wind about a spherical space. The retention frame portion may generally take the shape of two intersecting circles lying in different planes, two intersecting circles lying in different planes with inwardly curled ends, three intersecting circles lying in different planes, or a spherical spiral. In each of these examples, the retention frame portion can be stretched to the linear shape for deployment through a deployment instrument. The retention frame portion may wind about or through the spherical space, or other spheroid-shaped space, in a variety of other manners. One or both of the retention frame and retention frame lumen may be omitted, in which case the housing itself may assume or may be deformed into any retention shape described herein. Examples of alternative configurations are described in the U.S. patents and applications incorporated by reference herein.

The device may be inserted into a patient using a cystoscope or catheter. Typically, a cystoscope for an adult human has an outer diameter of about 5 mm and a working channel having an inner diameter of about 2.4 mm to about 2.6 mm. In embodiments, a cystoscope may have a working channel with a larger inner diameter, such as an inner diameter of 4 mm or more. Thus, the device may be relatively small in size. For example, when the device is elastically deformed to the relatively straightened shape suitable for insertion, the device for an adult patient may have a total outer diameter that is less than about 2.6 mm, such as between about 2.0 mm and about 2.4 mm. For pediatric patients, the dimensions of the device are anticipated to be smaller, e.g., proportional for example based on the anatomical size differences and/or on the drug dosage differences between the adult and pediatric patients. In addition to permitting insertion, the relatively small size of the device may also reduce patient discomfort and trauma to the bladder.

In one embodiment, the overall configuration of the device promotes in vivo tolerability in the bladder for most patients. In a particular embodiment, the device is configured for tolerability based on bladder characteristics and design specifications described in U.S. Pat. No. 8,679,094 (TB 112), which in pertinent part is incorporated herein by reference.

In one embodiment, the device may have a different dimension in at least two of the three directions, and in some cases in each of the three directions, so that the device is non-uniform in shape. Due to the non-uniform shape, the device may be able to achieve an orientation of reduced compression in the empty bladder, which also is non-uniform in shape. In other words, a particular orientation of the device in the empty bladder may allow the device to exert less contact pressure against the bladder wall, making the device more tolerable for the patient.

The overall shape of the device may enable the device to reorient itself within the bladder to reduce its engagement or contact with the bladder wall. For example, the overall exterior shape of the device may be curved, and all or a majority of the exterior or exposed surfaces of the device may be substantially rounded. The device also may be substantially devoid of sharp edges, and is exterior surfaces may be formed from a material that experiences reduced frictional engagement with the bladder wall. Such a configuration may enable the device to reposition itself within the empty bladder so that the device applies lower contact pressures to the bladder wall. In other words, the device may slip or roll against the bladder wall into a lower energy position, meaning a position in which the device experiences less compression.

The device also may be configured to facilitate buoyancy, such as with the use of low density materials of construction for the housing components and/or by incorporating gas or gas generating materials into the housing, as described for example in U.S. Patent Application Publication No. 2012/0089121 (TB 116), which in pertinent part is incorporated herein by reference.

The implantable drug delivery device can be made to be completely or partially bioerodible so that no explanation, or retrieval, of the device is required following release of the drug formulation. In some embodiments, the device is partially bioerodible so that the device, upon partial erosion, breaks into non-erodible pieces small enough to be excreted from the bladder. As used herein, the term “bioerodible” means that the device, or part thereof, degrades in vivo by dissolution, enzymatic hydrolysis, erosion, resorption, or combinations thereof. In one embodiment, this degradation occurs at a time that does not interfere with the intended kinetics of release of the drug from the device. For example, substantial erosion of the device may not occur until after the drug formulation is substantially or completely released. In another embodiment, the device is erodible and the release of the drug formulation is controlled at least in part by the degradation or erosion characteristics of the erodible device body. The devices described herein may be designed to conform to the characteristics of those described in U.S. Pat. No. 8,690,840 (TB 117), which is incorporated herein by reference.

Alternatively, the implantable drug delivery device may be at least partially non-bioerodible. It may be formed of medical grade silicone or polyurethane as known in the art or combinations of these materials. Other suitable materials of construction are envisioned. Following release of the drug, the device may be removed substantially intact or in multiple pieces.

Kits

The drug delivery devices described herein may be provided as part of kit, for example, so that the drug and/or the osmotic agent can be kept in a shelf-stable or storage suitable form. In one embodiment, as shown in FIG. 1, the kit 100 includes: (i) a drug delivery device 102 as described herein, including any combination of the disclosed or other suitable device features; (ii) a container 130 holding a substance, fluid, or precursor thereof 132 to be loaded into the device 102; and (iii) a means 134 for transferring the fluid component from the container 130 and into the drug delivery device 102 (e.g., into the elongated tube). As shown in FIG. 1, the kit 100 may include an ampoule 130 containing the fluid drug formulation 132.

In one embodiment, the fluid contained in the container is a fluid drug formulation to be delivered by the device. In another embodiment, the fluid is a precursor of the fluid drug formulation, such as a solvent for the drug, which dissolves the solid/semi-solid drug loaded in the elongated tube to form the fluid containing the drug. In one embodiment, as shown in FIG. 1, the means for transferring the fluid includes a device, such as a needle-and-syringe 134, as known in the art. In other embodiments, the means for transferring may include a pump, a funnel, a pipette, or the like.

In one embodiment, as shown in FIG. 3C, the kit also includes one or more pins 309 (i.e., closure devices) configured to be inserted into the aperture(s) through which the drug delivery device is filled and/or vented during filling. In one embodiment, the pin is constructed of a degradable material and dimensioned to be secured in the release structure after the fluid has been introduced into the elongated tube, such that upon insertion in vivo the degradable pin degrades to allow the drug-containing fluid to be released from the device via the release structure. The degradable pin may, for example, be made of poly(lactic acid) (PLA), poly(glycolic acid) (PGA), poly(lactide-co-glycolide) copolymers (PLGA), polydioxanone (PDS) or another biocompatible erodible material described herein or known in the art, or a combination thereof.

In one embodiment, as shown in FIG. 1, the device includes one or more air vents 115 as described above, and the kit 100 includes one or more plugs 116 configured to plug the air vents 115 upon introduction of the fluid, precursor, and/or water into the elongated tube and/or the water compartment. FIG. 2 shows an alternative configuration of a plug 216.

Methods

Various methods of using the osmotic drug delivery devices described herein to deliver one or more drugs to a patient are envisioned. The drug delivery devices may be any drug delivery device as described herein, including any suitable combination of the disclosed device features.

In one embodiment, a method of drug delivery includes deploying a drug delivery device into a patient's bladder via the patient's urethra, wherein the device includes a housing which defines a lumen and a fluid to be dispensed to the patient. The device may be elastically deformable between a first shape suitable for insertion through the urethra and a second shape suitable for retention of the device in the bladder. The device may be operable to move an osmotically-driven piston within the lumen to displace the fluid from the device. In a particular embodiment, the housing comprises an elongated tube, the piston comprises a gas, and the fluid comprises a drug.

In certain embodiments, the elongated tube has a first end with a release structure for releasing the fluid and an opposed second end and the housing further defines a reservoir that is connected to the second end of the elongated tube and in which an osmotic agent is disposed. The housing may include a water permeable wall for permitting water to enter the reservoir and contact the osmotic agent, and the piston may be operable to be advanced in the lumen toward the first end of the elongated tube under osmotic pressure generated by the osmotic agent to cause the fluid to be displaced out of the lumen via the release structure.

In certain embodiments, a method of drug delivery includes: (i) providing a drug delivery device that includes: (a) a first compartment configured to house a liquid; (b) a second compartment in communication with the first compartment and housing an osmotic agent, wherein at least a portion of a wall of the second compartment is water permeable; and (c) a liquid release structure in fluid communication with the first compartment; (ii) introducing the liquid into the first compartment, so that a fluid piston is formed between the liquid (and a drug contained therein) and the osmotic agent; (iii) inserting the drug delivery device into a patient, e.g., into the patient's bladder; and (iv) permitting water (e.g., from the site of insertion) to pass through the water permeable wall and into the second compartment (i.e., permitting water to be imbibed into the second compartment) via the water permeable wall. This thereby causes the second compartment to function as an osmotic pump, generating an osmotic pressure, which cause the fluid piston to be displaced, i.e., advanced, through the first compartment to drive the drug-containing liquid from the device, via the liquid release structure, and into the patient's body.

As shown in FIG. 1, the kit 100 may include a drug delivery device 102, a plug 116, a syringe with a needle 134, and an ampoule 130. The drug delivery device may be designed to be elastically bendable, and the system can be initially curved (e.g., in a retention shape), although it is shown straight in FIG. 1. The device of FIG. 1 has a fluid release structure 108 and an air vent 115, which will be closed with the plug 116 included with the kit 100.

In embodiments, at least a portion of the wall(s), e.g., the reservoir walls, surrounding the osmotic agent is water permeable. The osmotic agent may be in the form of one or more tablets. A water permeable region 112 is shown in the embodiment illustrated in FIG. 1. This water permeable region permits water to be imbibed into the system by osmosis. Any space between the orifice and osmotic agent may be initially void or air filled.

In one embodiment, introducing the fluid into the elongated tube includes injecting the fluid or precursor into the elongated tube via the release structure.

As shown in FIG. 3, the fluid 332 may be introduced into the elongated tube by a needle-type syringe 334, through the orifice 308. During fluid filling, the air vent 315 may remain open so that the fluid formulation cannot be expelled (by compressed air) when the syringe is pulled out after filling. After the fluid is loaded, a plug 316 may be used to close the air vent 314. That is, the method may include plugging an air vent in fluid communication with the elongated tube or the reservoir, after the fluid or precursor has been introduced into the elongated tube. For example, there may be a friction fit between the air vent and the plug, such that the fit is tight enough to endure the osmotic pressure in the device, which will occur once the device imbibes water. An alternative plug design is also shown in FIG. 2, where an air vent is made of elastomeric polymer and the plug with a bead at one end is stiff enough to be inserted and stay in the air vent. For example, the fluid may be introduced into the device by a physician or other medical personnel.

As shown in FIG. 3, the wall portion where the fluid formulation is loaded (e.g., the elongated tube) 307 may be substantially impermeable to the fluid drug formulation. The air shown between the fluid formulation and osmotic tablet is the air that is/becomes the gas piston 320. The wall of the device generally is sufficiently impermeable to air so that the air of the fluid piston remains within the lumen during the device operation. After the plug is inserted in the air vent, negative gauge pressure builds if the fluid formulation tends to flow out of the orifice by gravity not by osmosis, which may help prevent accidental expulsion of the fluid during the handling process.

Additionally, as shown in FIG. 3, a degradable pin 309 may be inserted into the release structure after the fluid or precursor has been introduced into the elongated tube, such that upon deployment of the device in the bladder the degradable pin degrades to allow the fluid containing the drug to be released from the device via the release structure. For example, a biodegradable pin may be inserted into the orifice, such as with a friction fit, to further decrease the risk of unwanted expulsion of the fluid during the handling and insertion process. The degradable pin can be made of a degradable material that dissolves relatively quickly (e.g., in less than a day) once in contact with water.

The device may then be inserted into a body lumen of a patient, where sufficient bodily fluid or water is available. For example, the device may be implanted in the bladder, where it comes into contact with urine. As shown in FIGS. 4A-4B, water 411 then becomes osmotically imbibed through the water permeable wall 412 of the reservoir, and the air of the gas piston 420 is compressed and advances through the elongated tube, such that pneumatic pressure is applied to the fluid formulation (drug/solvent), thereby causing the fluid formulation to be dispensed out of the orifice 408. The initial slug/bubble of air should be of an amount sufficient to separate the two fluid regions during device operation, even if a minor amount of the air may dissolve into either fluid or diffuse out through the wall of the device.

If there is a sufficient amount of osmotic agent loaded initially in the device, the concentration of the osmotic solution may remain constant as the agent is solubilized, although the osmotic influx region will increase. Therefore, the overall amount of osmotic water influx through the wall will increase over time as the osmotic influx region increases and the osmotic solution remains saturated. However, if there is an insufficient amount of osmotic agent loaded initially, the concentration of osmotic solution will decrease over time but the osmotic influx region will increase. Therefore, the multiplication of time-dependent osmotic solution concentration and the time-dependent osmotic influx region will determine how fast the osmotic solution pushes out drug fluid formulation.

In one embodiment, as shown in FIGS. 6A-6C, the drug delivery device includes a compartment 652 adjacent the reservoir 614, the compartment 652 being configured to house water 660 to be imbibed into the reservoir 614 via the water permeable portion 650 of the wall of the reservoir (e.g., a hydrophilic membrane positioned between the compartment and the reservoir). For example, this device embodiment may be suitable for implantation sites where sufficient bodily fluid or water is not available, such as in the uterus. In these embodiments, the method includes introducing water into the compartment via a port. In certain embodiments, the method also includes plugging an air vent in fluid communication with the compartment, after the water has been introduced into the compartment. Thus, the air vent may remain open during the filling process. The water injection port associated with the compartment may be left open so that negative gauge pressure cannot be generated as water in the compartment moves into the reservoir through the hydrophilic membrane.

In one embodiment, inserting the drug delivery device in the patient includes deploying the drug delivery device into the patient's bladder via the patient's urethra. For example, the device may be deployed through a deployment instrument, such as a catheter or cystoscope, positioned in a natural lumen of the body, such as the urethra, into a body cavity, such as the bladder. The deployment instrument typically is removed from the body lumen while the drug delivery device remains in the bladder or other body cavity for a prescribed treatment period. For example, the device may be implanted non-surgically and may deliver drug for several days, weeks, months, or more after the implantation procedure has ended.

The device, in some embodiments, may be deployed into the bladder of a patient in an independent procedure or in conjunction with another urological or other procedure or surgery, either before, during, or after the other procedure. In one embodiment, the device is implanted by passing the drug delivery device through a deployment instrument and releasing the device from the deployment instrument into the body. In cases in which the device is deployed into a body cavity such as the bladder, the device may assume a retention shape, such as an expanded or higher profile shape, once the device emerges from the deployment instrument into the cavity. The device may release one or more drugs that are delivered to local and/or regional tissues for therapy or prophylaxis, either peri-operatively, post-operatively, or both. The release may be controlled and may release the drug in an effective amount over an extended period. Thereafter, the device may be removed, resorbed, excreted, or some combination thereof. In certain embodiments, the device resides in the bladder releasing the drug over a predetermined period, such as two weeks, three weeks, four weeks, a month, or more. Thus, once implanted, the device may provide extended, continuous, intermittent, or periodic release of a desired quantity of drug over a desired, predetermined period. In certain embodiments, the device can deliver the desired dose of drug over an extended period, such as 12 hours, 24 hours, 5 days, 7 days, 10 days, 14 days, or 20, 25, 30, 45, 60, or 90 days, or more. The rate of delivery and dosage of the drug can be selected depending upon the drug being delivered and the disease or condition being treated.

In one embodiment, as shown in FIGS. 3A-3C, the fluid is a solution of the drug. In another embodiment, as shown in FIGS. 5A-5C, the drug delivery device includes a solid or semi-solid formulation of the drug 531 housed within the elongated tube, and the fluid precursor 533 is a solvent for the drug, such that upon introduction of the fluid precursor into the elongated tube, the fluid precursor dissolves the drug to form the fluid containing the drug to be driven from the device. For example, if the drug is more stable and/or displays improved handling in a solid form than in a liquid form, or if there are safety issues associated with handling the drug, the device may be pre-loaded with a solid form of the drug.

The device may be used to treat interstitial cystitis, radiation cystitis, pelvic pain, overactive bladder syndrome, bladder cancer, neurogenic bladder, neuropathic or non-neuropathic bladder-sphincter dysfunction, infection, post-surgical pain or other diseases, disorders, and conditions treated with drugs delivered to the bladder. The device may release drug locally to the bladder and regionally to other sites near the bladder. The device may deliver drugs that improve bladder function, such as bladder capacity, compliance, and/or frequency of uninhibited contractions, that reduce pain and discomfort in the bladder or other nearby areas, or that have other effects, or combinations thereof. The bladder-deployed device also may deliver a therapeutically effective amount of one or more drugs to other genitourinary sites within the body, such as other locations within urological or reproductive systems of the body, including the kidneys, urethra, ureters, penis, testes, seminal vesicles, vas deferens, ejaculatory ducts, prostate, vagina, uterus, ovaries, or fallopian tubes, among others or combinations thereof. For example, the drug delivery device may be used in the treatment of kidney stones or fibrosis, erectile dysfunction, among other diseases, disorders, and conditions. The drug may include gemcitabine, oxaliplatin, and/or another chemotherapeutic agent, trospium and/or another antimuscarinic agent, or lidocaine and/or another anesthetic agent.

Subsequently, the device may be retrieved from the body, such as in cases in which the device is non-resorbable or otherwise needs to be removed. Retrieval devices for this purpose are known in the art or can be specially produced. The device also may be completely or partially bioerodible, resorbable, or biodegradable, such that retrieval is unnecessary, as either the entire device is resorbed or the device sufficiently degrades for expulsion, for example, from the bladder during urination. The device may not be retrieved or resorbed until some of the drug, or preferably most or all of the drug, has been released. If needed, a new drug-loaded device may subsequently be implanted, during the same procedure as the retrieval or at a later time.

The present invention may be further understood with reference to the following non-limiting examples.

### EXAMPLES

Embodiments of the devices disclosed herein were manufactured and tested. In one example, a silicone tube having a length of 10 cm (1.02 mm ID×2.16 mm OD) was connected to a hydrophilic HP-93A-100 tube (2.64 mm ID×3.05 mm OD) filled with NaCl tablets in an amount of 230 mg/2.3 cm. In another example, a silicone tube having a length of 13.5 cm (0.51 mm ID×0.94 mm OD) was connected to a hydrophilic HP-93A-100 tube (2.64 mm ID×3.05 mm OD) filled with NaCl tablets in an amount of 241 mg/2.2 cm. The silicone tubes were filled with a methylene blue (MB) aqueous solution and immersed in degassed DI water. Based on visual observations, the aqueous solution was advanced by water flux into the hydrophilic tube, while the air slug served as a piston or separator.

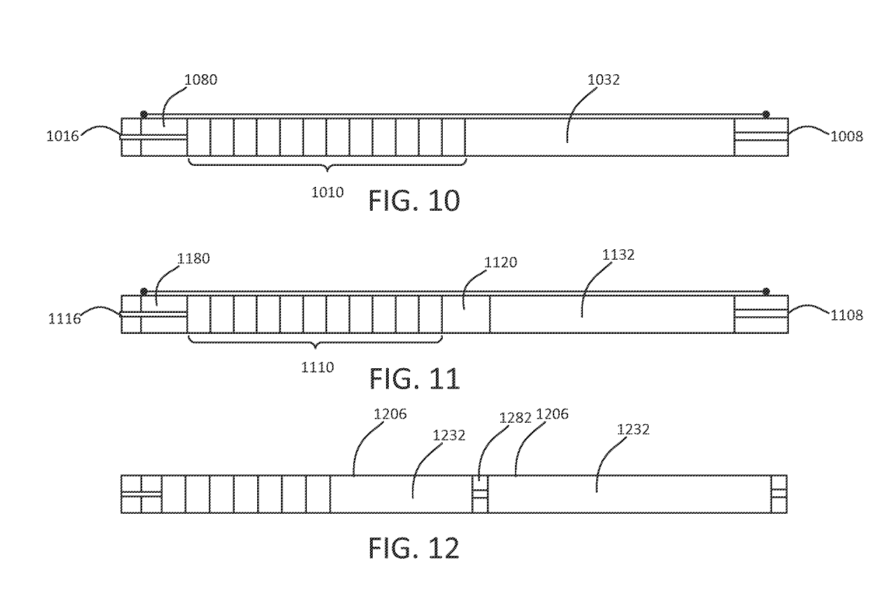

In another example, gemcitabine (drug) and trisodium citrate (osmotic agent) release profiles for units with and without an air bubble (i.e., air gap or fluid piston) between the solid and liquid sections were tested. Devices similar to those shown in FIGS. 10-11 were manufactured and tested.

Specifically, devices containing ˜7.5 cm solid 93% trisodium citrate tablets (1010, 1110) and ˜15 cm liquid gemcitabine HCl in water (5 mg FBE/mL) (1032, 1132) were manufactured. An air vent spacer orifice 5 mm in length and having a 500 μm orifice ID (1080, 1180) was secured at the tube end adjacent the osmotic tablets with silicone adhesive. A solution injection/release spacer orifice 5 mm in length and having a 300 μm orifice ID (1008, 1108) was secured at the opposing tube end with silicone adhesive. The gemcitabine solution was injected through the injection orifice with a syringe. A nitinol pin (1016, 1116) was fit into the air vent spacer orifice after the drug solution was injected into the unit. Three units in which the liquid drug solution and osmotic tablets were touching were prepared (as shown in FIG. 10), as well as three units in which an air slug ˜2 cm in length (1120) was provided between the osmotic tablets and the liquid drug solution (as shown in FIG. 11). The units were placed in 100 mL deionized water at 37° C. and the release media was mixed by pipetting 5 mL out/in three times before each measurement sample was taken.

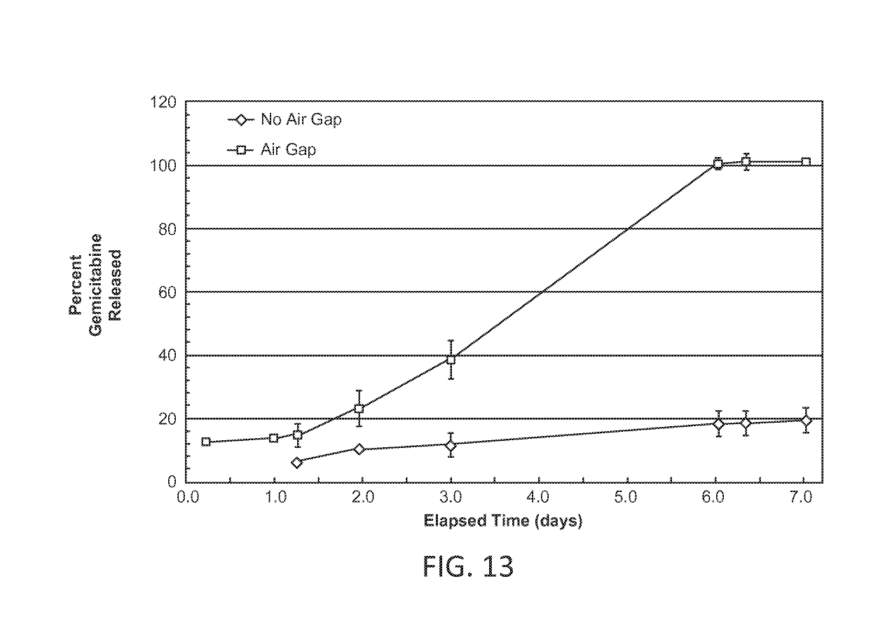

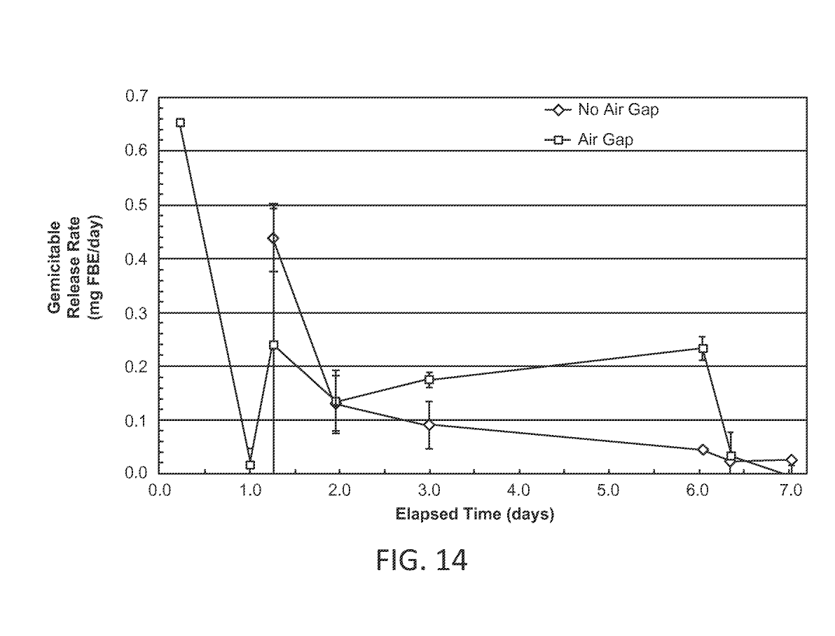

FIGS. 13-16 show the results of these tests. FIG. 13 shows the percent gemcitabine released measured over time, while FIG. 14 shows the gemcitabine release rate over time, for the units having the air gap versus the units with no air gap. As shown, the air gap units immediately began releasing the gemcitabine after the devices were immersed in the water, while the units without the air gap did not begin releasing drug until after 24 hours. Surprisingly, FIG. 13 shows that the units with the air gap are able to release 100% of the gemcitabine solution, compared to the units without an air gap, which release only about 20% of the gemcitabine solution. Moreover, the air gap units have a higher gemcitabine release rate at later time points, which is desirable for drugs for which extended release profiles are desirable.

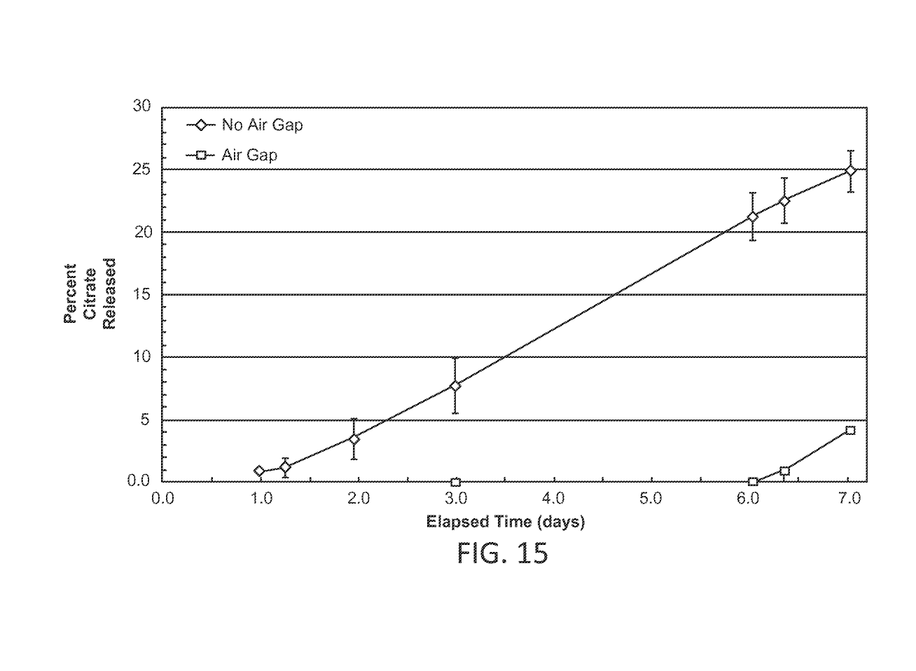

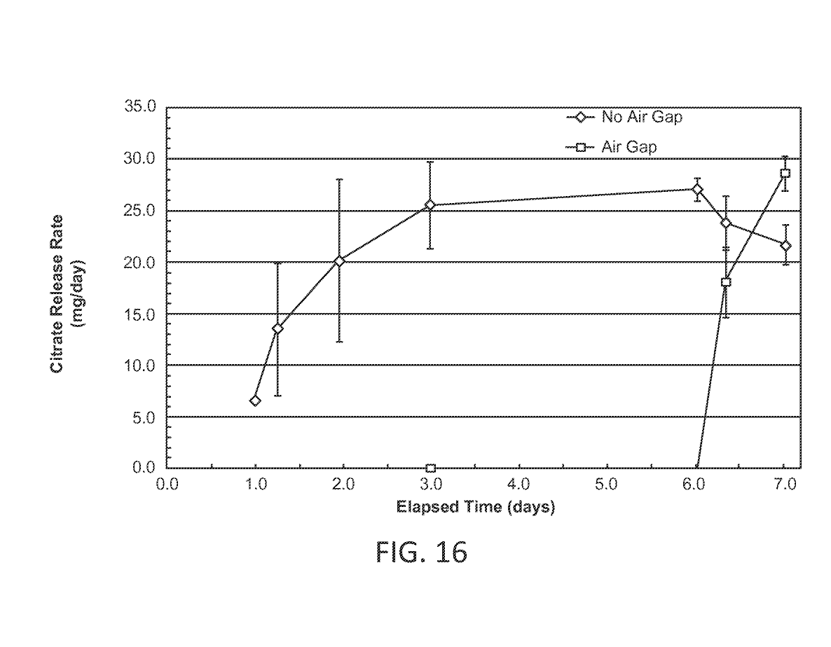

FIG. 15 shows the percent citrate (osmotic agent) released over time, while FIG. 16 shows the citrate release rate over time, for the units having the air gap versus the units with no air gap. Generally, FIGS. 13-16 show that the air gap is maintained between the osmotic tablets and the drug solution for seven days. Moreover, the air gap prevents release of the osmotic agent until 100% of the gemcitabine is released. Thus, the air gap is acts as a piston and keeps the osmotic and drug sections separate during drug release, leading to a higher percentage of gemcitabine release and higher release rates at later days.

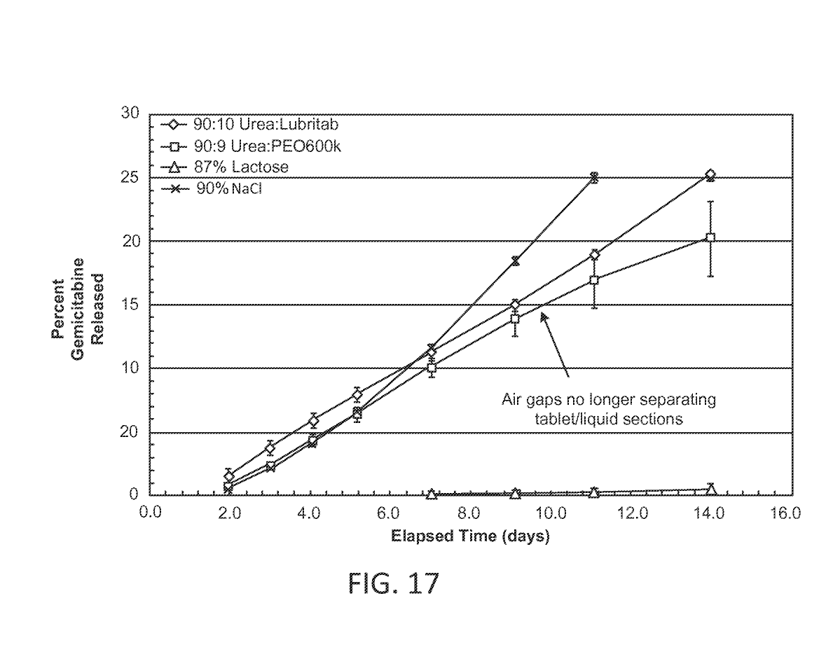

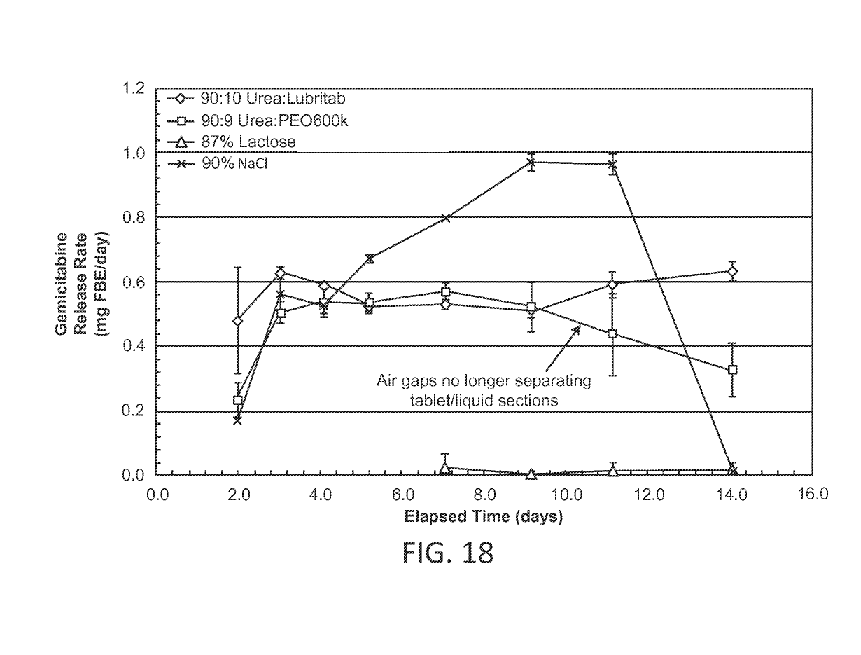

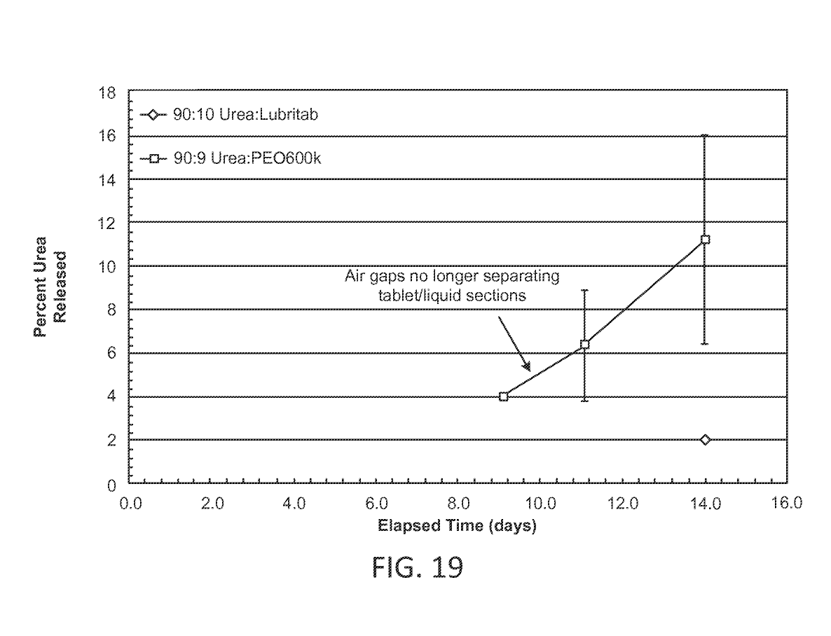

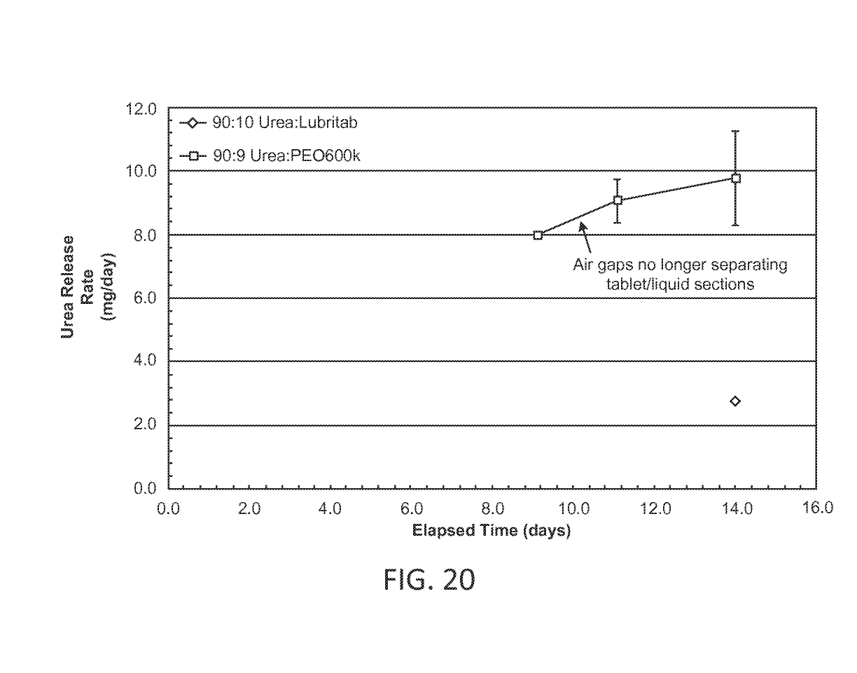

In another example, units with an air gap between the solid and liquid sections were manufactured with different osmotic agents, to determine how changing the osmotic agent in the tablets changes the gemcitabine release profile. Devices similar to those prepared for the tests reported on in FIGS. 13-16 were prepared, but with a longer liquid core length of ˜30 cm. The osmotic agents tested included: (1) 90% urea, 10% Lubritab® (J. Rettenmaier & Sane GmbH+Co. KG); (2) 90% urea, 9% poly(ethylene oxide) having an average molecular weight of 600,000 (“PEO(600K)”), 0.5% Neusilin® UFL2 (Fuji Chemical Industry Co., Ltd), 0.5% magnesium stearate; (3) 87% lactose monohydrate, 8% polyethylene glycol (PEG) having an average molecular weight of 8,000, 5% Plasdone™ K-29/32 (ISP Pharmaceuticals); and (4) 90% NaCl, 10% Polyplasdone™ XL10 (ISP Pharmaceuticals). FIG. 17 shows the percent gemcitabine released over time for the devices, while FIG. 18 shows the gemcitabine release rate over time for the devices. FIG. 19 shows the percent urea released over time for the two devices containing urea in the osmotic agent, while FIG. 20 shows the urea release rate over time for the two devices containing urea in the osmotic agent.

As shown in FIGS. 17-20, the Urea:Lubritab® devices show an approximately constant gemcitabine flux from day 3 to day 14, with 100% gemcitabine released at day 14. The Urea:Lubritab® results also indicate that the urea dilution is offset by increased surface area. The Urea:PEO(600K) devices show an approximately constant gemcitabine flux from day 3 to day 7. The Urea:PEO(600K) also indicate that the urea dilution is initially offset by increased surface area. However, the results indicate that the air gaps were not maintained between the gemcitabine and Urea:PEO(600K), which could be due to a decrease in surface tension by the PEO. The lactose devices show a very long lag time in drug release. The NaCl devices show an increase in gemcitabine flux from day 2 to day 9 and a constant gemcitabine flux from day 9 to day 11, with 100% gemcitabine released at day 11.

These examples show that the air gap devices significantly outperform similar devices having no air gap. The air gap devices are capable of releasing 100% of their liquid drug payload at a constant or increasing release rate. Thus, drug delivery devices may be tailored based on the desired drug release profile.

These devices advantageously are capable of delivering drugs that are available in liquid form only or drugs that are more stable/safe in solid form for storage. That is, these devices allow the drug to be formulated for optimum stability/solubility, without changing the osmotic behavior or release rate of the device. Moreover, these devices solve the problems associated with known rigid osmotic drug delivery devices, by providing a flexible, substantially frictionless piston. This piston design allows the device body to be made of flexible elastomeric materials, because the flexible piston is able to follow the contour of a device having kinks and/or curvatures along its length. In contrast, a flexible device having a rigid piston was found to experience leakage at the piston because the housing would inflate due to the osmotic pressure behind the piston. The flexible device may also advantageously be used in a wider variety of applications and insertion/implantation sites than a rigid device.

Publications cited herein and the materials for which they are cited are specifically incorporated by reference. Modifications and variations of the methods and devices described herein will be obvious to those skilled in the art from the foregoing detailed description. Such modifications and variations are intended to come within the scope of the appended claims.

1. A medical device comprising:
a housing defining a lumen; and
an osmotically-driven piston moveable within the lumen,
wherein the housing is elastically deformable between a first shape suitable for insertion through a patient's urethra and a second shape suitable for retention of the device in the patient's bladder, and
wherein the piston is a fluid which, in use, is configured to separate a substance to be dispensed from the device from an osmotic solution on a driving side of the piston.

2. The device of claim 1, further comprising a substance to be dispensed to the patient, wherein the device is operable to move the piston within the lumen to displace the substance from the device.

3. The device of claim 2, wherein:
the housing comprises an elongated tube,
the piston comprises a gas, and
the substance comprises a drug.

4. The device of claim 3, wherein:
the elongated tube comprises a first end having a release structure for releasing the substance and an opposed second end,
the housing in further defines a reservoir that is connected to the second end of the elongated tube and which an osmotic agent is disposed,
the housing further comprises a water permeable wall for permitting water to enter the reservoir and contact the osmotic agent, and
the piston is operable to be advanced in the lumen toward the first end of the elongated tube under osmotic pressure generated by the osmotic agent to cause the substance to be displaced out of the lumen via the release structure.

5. The device of claim 1, wherein the housing comprises an annular tube having a single, central lumen.

6. The device of claim 5, wherein the central lumen has a diameter from 1 mm to 3 mm.

7. The device of claim 3, wherein the elongated tube is formed of an elastomeric polymer.

8. The device of claim 7, wherein the elastomeric polymer is substantially water and gas impermeable or has a coating that is substantially water and gas impermeable.

9. The device of claim 7, wherein the elastomeric polymer comprises silicone or polyurethane.

10. The device of claim 9, wherein the tube comprises silicone coated with parylene.

11. The device of claim 4, wherein the reservoir is formed by an annular tube connected to or integrally formed with the elongated tube which contains the substance.

12. The device of claim 11, wherein the water permeable wall comprises a water permeable disc at an end of the annular tube.

13. The device of claim 4, wherein the water permeable wall of the housing comprises a hydrophilic polymer.

14. The device of claim 12, wherein the hydrophilic polymer comprises a thermoplastic polyurethane.

15. The device of claim 4, wherein the osmotic agent is in a solid form.

16. The device of claim 15, wherein the osmotic agent is in the form of one or more tablets.

17. The device of claim 4, wherein the osmotic agent is selected from the group consisting of monosodium citrate, disodium citrate, trisodium citrate, lactose, sodium chloride, urea, sucrose, and combinations thereof.

18. The device of claim 1, further comprising a retention frame which urges the device into the second shape, which second shape comprises a coil, in the absence of a compressive load needed to deform the device into the first shape.

19. The device of claim 3, wherein the drug comprises gemcitabine, oxaliplatin, and/or another chemotherapeutic agent.

20. The device of claim 3, wherein the drug comprises oxybutynin, trospium and/or another antimuscarinic agent.

21. The device of claim 3 wherein the drug comprises lidocaine and/or another anesthetic agent.

22. The device of claim 3, wherein the elongated tube has an inner diameter sized such that capillary force is dominant over gravitational force within the tube.

23. The device of claim 4, wherein the release structure comprises an aperture and/or a check valve.

24. The device of claim 4, further comprising a connector connecting the elongated tube and the reservoir.

25. The device of claim 1, wherein the piston is a bubble of air or another gas.

26. The device of claim 1, wherein:
the housing comprises an elongated tube having a first end comprising a release structure for releasing a fluid and an opposed second end, the elongated tube being configured to receive a fluid drug or a precursor thereof,
the housing further defines a reservoir that is connected to the second end of the elongated tube and in which an osmotic agent is disposed,
the housing further comprises a water permeable wall for permitting water to enter the reservoir and contact the osmotic agent,
the device is configured such that upon receipt of the fluid or precursor thereof, the piston comprises a gas formed between the fluid and the osmotic agent, and
the device is configured to imbibe water into the reservoir via the water permeable wall to advance the gas piston through the elongated tube via osmotic pressure generated by the osmotic agent to drive the fluid from the device via the release structure.

27. The device of claim 26, further comprising an air vent in fluid communication with the elongated tube or the reservoir, the air vent being configured to be plugged once the elongated tube receives the fluid or precursor thereof.

28. The device of claim 26, wherein the device is configured to receive the fluid or fluid precursor via the release structure.

29. The device of claim 26, wherein:
the device further comprises a solid or semi-solid formulation of the drug, which is housed within the elongated tube, and
the fluid precursor is a solvent for the drug, such that upon receipt of the fluid precursor in the elongated tube, the fluid precursor dissolves the drug to form the fluid driven from the device.

30. The device of claim 29, wherein the solvent comprises water or dimethyl sulfoxide.

31. The device of claim 26, further comprising a compartment adjacent the reservoir, the compartment being configured to house water to be imbibed into the reservoir via the water permeable wall.

32. The device of claim 31, wherein the water permeable wall of the reservoir comprises a hydrophilic membrane positioned between the reservoir and the compartment.

33. The device of claim 31, further comprising an air vent in fluid communication with the compartment, the air vent being configured to be plugged once the compartment receives the water.

34. A kit comprising:
the device of claim 3;
a container housing the substance, the fluid, or a precursor thereof; and
a device for transferring the fluid or precursor from the container and into the elongated tube.

35. The kit of claim 34, wherein:
the device further comprises a solid or semi-solid formulation of the drug, which is housed within the elongated tube, and
the container houses a precursor comprising a solvent for the drug.

36. The kit of claim 34, further comprising a degradable pin configured to be inserted into the release structure after the substance, fluid, or precursor has been introduced into the elongated tube, such that upon insertion of the device into the bladder the degradable pin degrades to allow the fluid to be released from the device via the release structure.

37-38. (canceled)

39. A method of drug delivery, comprising:
deploying a drug delivery device into a patient's bladder via the patient's urethra, the device comprising a housing defining a lumen and a fluid to be dispensed to the patient,
wherein the device is elastically deformable between a first shape suitable for insertion through the urethra and a second shape suitable for retention of the device in the bladder,
wherein the device is operable to move an osmotically-driven piston within the lumen to displace the fluid from the device.

40. The method of claim 39, wherein:
the housing comprises an elongated tube,
the piston comprises a gas, and
the fluid comprises a drug.

41-50. (canceled)
